(19) United States
(12) Patent Application Publication
(10) Pub. No.: US 2015/0128354 A1
(43) Pub. Date: May 14, 2015

(54) METHODS, CIRCUITS, APPARATUSES AND ASSEMBLIES FOR PROVIDING A MATTRESS OR BODY PORTION SUPPORT CUSHION WITH A SENSOR LAYER

(71) Applicant: ENHANCED SURFACE DYNAMICS, INC., WELLESLEY, MA (US)

(72) Inventors: Lior Greenstein, Tel-Aviv (IL); Amir Ben Shalom, Modiin (IL); John S. Ehlinger, Wellesley, MA (US); Efrat Zaritzky, Tel-Aviv (IL)

(73) Assignee: ENHANCED SURFACE DYNAMICS, INC., WELLESLEY, MA (US)

(21) Appl. No.: 14/395,516

(22) PCT Filed: Apr. 11, 2013
(86) PCT No.: PCT/IB13/52878

§ 371 Date: Oct. 20, 2014

Related U.S. Application Data
(60) Provisional application No. 61/625,876, filed on Apr. 18, 2012.
(60) Provisional application No. 61/769,977, filed on Feb. 27, 2013.

(51) Int. Cl. A61G 7/057; A61B 5/00; G05B 15/02; A61G 5/10

(52) U.S. Cl. CPC A61G 7/05776; A61G 5/1043; A61B 5/445; G05B 15/02; A61M 2205/3303; A61G 2203/34; A61G 2203/46
USPC 5/710

(57) ABSTRACT

A resting body support, such as a mattress or an encasement for a resting body support, comprising a sensor layer is disclosed which may incorporate a mattress adjustment system. The system may comprise at least one pressure detection mat comprising a plurality of sensors arranged in a matrix and at least one processor configured to monitor a plurality of pressure values detected by each of said sensors; and at least one active mattress comprising a plurality of cells, the cells being controlled by an inflation control unit that is connected to said processor, and operable to individually adjust the level of inflation of said plurality of cells based on said plurality of pressure values.

This application claims priority and benefit from U.S. Provisional Patent Application 61/625,876, filed Apr. 18, 2012 and U.S. Provisional Patent Application 61/769,977, filed Feb. 27, 2013, the contents and disclosures of which are incorporated by reference in their entirety.

### FIELD OF THE INVENTION

The present invention relates to pressure sensors. More specifically, the present invention relates to methods, circuits, devices, apparatuses, encasements and systems for providing a pressure sensor layer to a mattress or other body portion support structure.

### BACKGROUND

Pressure-wounds such as decubitus ulcers, which are commonly known as pressure ulcers or bedsores, are lesions developed when a localized area of soft tissue is compressed between a bony prominence and an external surface for a prolonged period of time. Pressure ulcers may appear in various parts of the body, and their development is affected by a combination of factors such as unrelieved pressure, friction, shearing forces, humidity and temperature.

Currently, about 10%-15% of hospitalized patients are estimated to have bedsores at any one time (Medicare website 2009). However, it is not only hospitalized patients who suffer from pressure-wounds: for example, people confined to wheelchairs are prone to suffer from pressure-wounds, especially in their pelvis, lower back and ankles. Although easily prevented and completely treatable if found early, bedsores are painful, and treatment is both difficult and expensive. In many cases bedsores can prove fatal—even under the auspices of medical care.

The most effective way of dealing with pressure-wounds is to prevent them. Existing preventive solutions are either passive (e.g. various types of cushioning) or active, including a range of dynamic mattresses that alternate the inflation/deflation of air cells. Pressure relief mattresses however tend to re-distribute pressure also from locations where there was no need to relieve pressure thereby needlessly creating higher pressure in sensitive areas. Moreover, such mattresses are typically designed for patients lying down in hospital beds, and hardly answer the needs of individuals who spend considerable amounts of time sitting up, confined to a wheelchair or the like.

The most common preventive approach is keeping a strict care routine of relieving pressure off sensitive body areas of a patient every 2-3 hours. This can be done with patients under strict medical care. As well as being a difficult, labor intensive and costly task, such a care routine does not meet the needs of independent individuals who do not require ongoing supervision of a caretaker, such as paraplegics who use a wheelchair for mobility.

The need remains, therefore, for a reliable, cost effective system and method for preventing the development of pressure-wounds. Embodiments described hereinbelow address this need.

### SUMMARY

It is an aspect of the current disclosure to introduce a mattress adjustment system comprising at least one pressure detection mat comprising a plurality of sensors arranged in a matrix and at least one processor configured to monitor a plurality of pressure values detected by each of the sensors; and at least one active mattress comprising a plurality of cells, the cells being controlled by an inflation control unit that is connected to the processor, and operable to individually adjust the level of inflation of the plurality of cell based on the plurality of pressure values.

Optionally, the pressure detection mat comprises at least one layer of an insulating material sandwiched between a first layer of conducting strips and a second layer of conducting strips, the conducting strips of the first layer and the strips of the second layer overlapping at a plurality of intersections, each of the sensors comprising at least one of the intersections; a driving unit configured to supply electrical potential selectively to the conducting strips of the first layer; and a control unit wired to the conductive strips of the second layer and operable to control the driving unit; the processor being further configured to monitor an electrical potential on the conductive strips of the second layer, to calculate a plurality of impedance values for each of the intersections and to determine a plurality of pressure values for each of the sensors based on the impedance values.

Where appropriate, the processor may be configured to detect a risk locus comprising an area corresponding to one or more of the sensors determined to have a pressure value beyond a predefined threshold pressure for over a predefined duration; and the inflation control unit may be operable to individually adjust the level of inflation of the plurality of cells in order to redistribute the pressure at the risk locus. It is noted that where required, the predefined duration may be instantaneous. Alternatively, the predefined duration may be dependent on the predefined threshold pressure, wherein the area corresponding to the sensor detecting a higher predefined threshold pressure requires a shorter predefined duration for an area corresponding to the sensor to be detected as a risk locus.

Where appropriate, the redistribution of pressure may result in one or more pressure redistribution targets selected from the group consisting of: pressure values measured by the sensors corresponding to the risk locus are lowered such that the risk locus is no longer designated as a risk locus, the pressure measured by the sensors corresponding to the risk locus are lowered to zero, and other loci outside the risk locus not becoming designated as a risk locus as a result of the pressure redistribution.

Optionally, the redistribution of pressure may be one or more adjustment patterns selected from the group consisting of: reducing the pressure at the risk locus; increasing the pressure of a peripheral area neighboring the risk locus; and reducing the pressure of a peripheral area neighboring the risk locus. Optionally, the adjustment pattern may change over time.

Variously, each cell may cover an area corresponding to the area covered by a plurality of sensors. Additionally or alternatively, the cells are identical in size and shape. Where required, the cells may be of more than one size.

Optionally, the cells may be incorporated in at least one portion of the mattress designed to support a body part vulnerable to bedsore formation are smaller than the cells in other portions of the active mattress. Where appropriate, the body part vulnerable to bedsore formation is one or more of a selection from the group consisting of: a sacrum, a coccyx, a heel, a hip, an elbow, a knee, an ankle or the back of the cranium

According to another aspect of the disclosure, a method is taught for preventing the development of pressure-wounds. The method may comprise steps including: providing at least one pressure detection mat comprising a plurality of sensors configured to detect pressure; providing at least one active mattress comprising a plurality of cells operable to adjust its inflation; collecting data from the plurality of sensors; analyzing the data; detecting a risk locus; and adjusting the level of inflation of one or more cells to redistribute the pressure at the risk locus.

Optionally, the steps of collecting data from the plurality of sensors; analyzing the data; detecting a risk locus; and adjusting the level of inflation of one or more cells to redistribute the pressure at the risk locus may be repeated as an adjustment cycle. Accordingly, the adjustment cycle may be characterized by a predetermined rate. Alternatively or additionally, the adjustment cycle may be repeated for a predetermined number of times or a predetermined amount of time.

Optionally, the adjustment cycle may be started and stopped according to start/stop instructions provided by the subject or a user. Alternatively or additionally, each adjustment cycle may be initiated by the subject or a user.

Variously, the redistribution of pressure results in one or more pressure redistribution targets selected from the group consisting of: pressure values measured by the sensors corresponding to the risk locus are lowered such that the risk locus is no longer designated as a risk locus; the pressure measured by the sensors corresponding to the risk locus are lowered to zero; other loci outside the risk locus not becoming designated as a risk locus as a result of the pressure redistribution.

Optionally the method may further comprise the step of displaying a visual output based upon the data. Optionally again, the method may further comprise the step of storing the data in at least one data storage unit.

It is therefore according to an aspect of the disclosure to introduce a sensor-integrated resting body support comprising a resting body support and a sensor layer. The sensor-integrated resting body support may comprise one or more sensors; and one or more sensor support and connectivity elements adapted to electrically connect to the one or more sensors and to provide the one or more sensors with electrical connectivity to an external interface. Variously, the sensor layer may be discrete and embedded into the resting body support. Additionally or alternatively, the sensor layer may be integral with an inner layer of the resting body support

Optionally, the one or more sensors are of a type selected from the group consisting of pressure sensors, temperature sensors, humidity sensors, and vital sign sensors. Accordingly, the one or more sensors may be pressure sensors, which may comprise: at least one layer of an insulating material sandwiched between a first layer of conducting strips and a second layer of conducting strips, the conducting strips of the first layer and the strips of the second layer overlapping at a plurality of intersections, each of the sensors comprising at least one of the intersections; a driving unit configured to supply electrical potential selectively to the conducting strips of the first layer; a control unit wired to the conductive strips of the second layer and operable to control the driving unit; and a processor configured to monitor an electrical potential on the conductive strips of the second layer, to calculate a plurality of impedance values for each of the intersections and to determine a plurality of pressure values for each of the sensors based on the impedance values.

According to various embodiments, the resting body support may be selected from a group consisting of a mattress, a seat, a cushion, a backrest, a handrail, a footrest or the like as well as combinations thereof.

In another aspect of the disclosure a sensor-integrated resting body support encasement is presented comprising a resting body support encasement and a sensor layer comprising: one or more sensors; and one or more sensor support and connectivity elements adapted to electrically connect to the one or more sensors and to provide the one or more sensors with electrical connectivity to an external interface.

Variously, the sensor layer may be connected with an inner layer of the resting body support encasement. Alternatively, or additionally, the sensor layer may be discrete and detachably connected to an inner surface of the resting body support encasement. Alternatively, or additionally, again, the sensor layer is discrete and embedded into the resting body support encasement. Optionally, the sensor layer may be integral with an inner layer of the resting body support encasement and deposited onto the resting body support encasement.

Where appropriate, one or more sensors may be of a type selected from the group consisting of pressure sensors, temperature sensors, humidity sensors, and vital sign sensors.

### BRIEF DESCRIPTION OF THE FIGURES

For a better understanding of the invention and to show how it may be carried into effect, reference will now be made, purely by way of example, to the accompanying drawings.

With specific reference now to the drawing in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention; the description taken with the drawing making apparent to those skilled in the art how the several forms of the invention may be embodied in practice.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

In the accompanying drawings,

FIG. 1a shows the main components of a general embodiment of a pressure-wound prevention system;

FIG. 1b shows a extended pressure-wound prevention system including a plurality of pressure-wound prevention sub-systems of different kinds;

FIG. 2a shows a cross section of an embodiment of a single sensor; and

FIGS. 2b-e show various isometric projections of embodiments of a pressure-detection sheet;

FIGS. 3a-b show a top view and a section through view of a further embodiment of a pressure detection sheet;

FIG. 4a shows a top perspective view of an exemplary sensor layer, including sensors such as pressure sensors, according to embodiments;

FIG. 4b shows a cross-sectional view of an exemplary sensor layer incorporated into a mattress according to embodiments;

FIG. 5a shows a perspective view with cutaway of an exemplary mattress with an integrated sensor layer according to embodiments;

FIG. 5b shows a perspective view with cutaway of an exemplary mattress within an exemplary encasement having an integrated sensor layer according to embodiments;

FIG. 6 showing construction steps and elements of an exemplary mattress with integrated sensor layer according to embodiments.

FIG. 7 showing construction steps of an exemplary mattress encasement with an integrated sensor layer according to embodiments.

FIG. 8 shows a mattress adjustment system having a pressure detection mat with an active mat.

FIGS. 9a-f shows various representations of patterns of inflation adjustments of cells in the active mat.

FIGS. 10a-e shows various embodiments of the active mat.

FIG. 11 shows a pressure-wound prevention system incorporated into a wheelchair;

FIGS. 12a-d show various representations of how pressure data may be displayed on a screen of an embodiment of display system; and

FIGS. 13a-b are flowcharts of methods for preventing the development of pressure wounds.

### DETAILED DESCRIPTION

Embodiments of the pressure detection system and method described hereinbelow are directed towards preventing pressure-wounds from developing in a subject. The embodiments generally provide a caretaker with indications of pressure distribution and ongoing, accumulated pressure exerted upon body parts of a subject, which may result in the creation or progression of a bedsore. A caretaker may then take appropriate action, such as to move the subject or change his cushioning in a way that relieves pressure upon the effected body part. Embodiments of the system may also be used for ongoing analysis and recording of a subject's care routine.

The applicants' copending international patent application WO 2010/119441, which is incorporated herein by reference, discloses a pressure sensing system for use in preventing decubitus ulcers, or bedsores, which comprises a sensing-mat including a plurality of sensors configured to detect pressure applied to body parts of a subject resting on a surface such as a bed or a chair. Information received from the sensors is analyzed by the system, which further issues alerts according to sensor readings.

It will be appreciated that embodiments of the pressure-detection system allow a caretaker to move the patient only when it is needed. Furthermore, attention may be targeted towards the pressured part of the body specifically, which may be repositioned or cushioned as required. It is further noted that embodiments of such a system may further assist in monitoring a subject's care routine and his caretaker's performance.

Various embodiments of the system and method for preventing pressure-wounds are presented hereinbelow. Typically, they utilize pressure-detection elements to determine which areas of a subject's body are at risk of developing pressure ulcers. One of these elements could be a pressure-detection sensing mat, configured to couple with a pressure-wound prevention system as outlined below.

### Pressure-Wound Prevention System Including a Sensing Mat

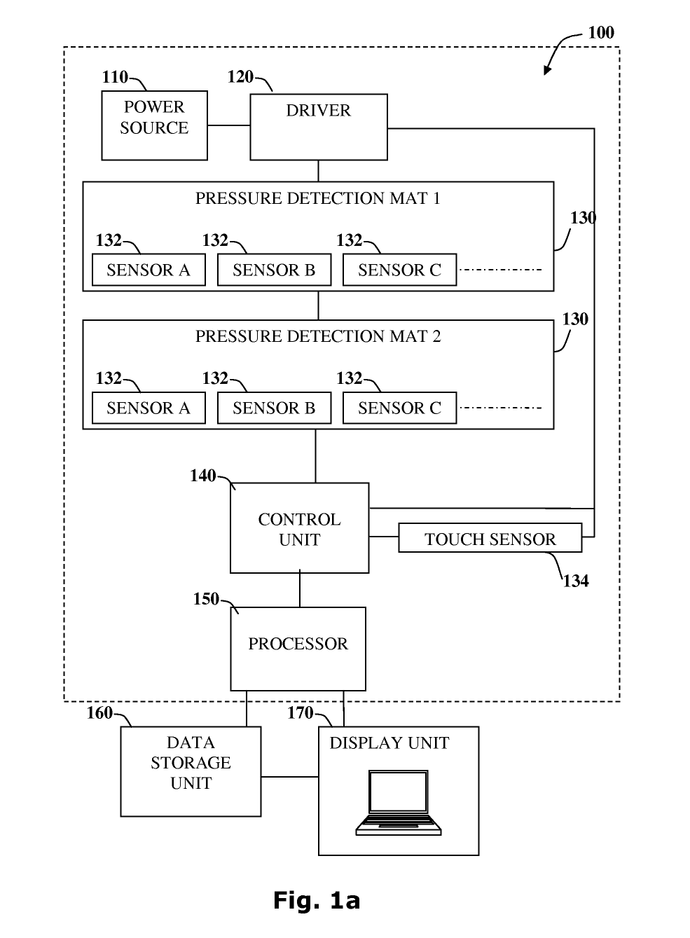

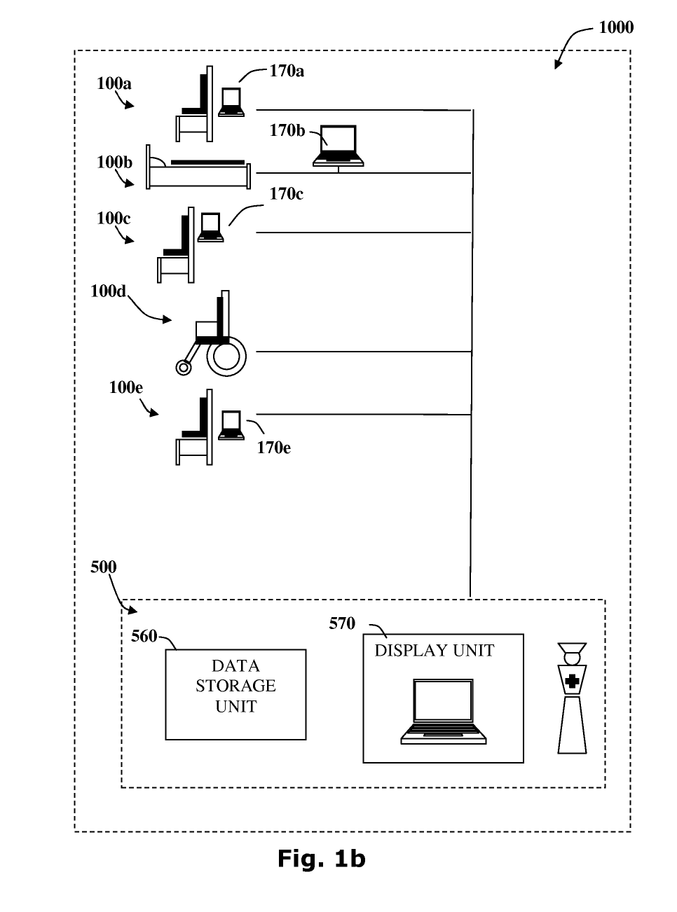

Reference is now made to the block diagram of FIG. 1a, representing the main components of a general embodiment of a pressure-wound prevention system 100. Embodiments of such a system may include at least one pressure-detection mat 130 comprising a plurality of sensors 132, a driver 120, a control unit 140 typically connected to a power source 110, a processor 150, a data storage unit 160 and a display system 170. The system may optionally include additional sensors such as a touch sensor 134 configured to detect contact between a platform and a subject's body. In this embodiment, the driver 120 selectively supplies voltage to sensors in the pressure-detection mat and optionally to the touch sensor 134. The processor 150 monitors the potential across the sensors in the pressure detection mat, calculates impedance values for each sensor, and stores that data in a data storage unit 160. The processor optionally monitors data received from the touch sensor as well. The stored data may be further processed, analyzed, and displayed on a display system 170, such as computer screens, laptops, PDAs, cellular phone screens, printed sheets, integrated LCD screens (e.g. Thin Film Transistors, touch screens) and the like. Although presented in the block diagram as separate blocks, the system may optionally be integrated into a stand-alone system.

Measurement readings from the multiple sensors of the pressure-detection mat may be transmitted to a processor 150. Data transmission may be wireless or via data cables according to requirements. The processor 150 may be configured to interpret impedance values and to analyze the data to determine which sensors had pressure applied to them. The interpretation may be performed by consulting with a lookup table which maps impedance values at a given frequency to pressure values, typically in units of millimeters of mercury, as commonly used in medical settings, although other pressure units such as pascals, atmospheres, pounds per square inch or the like may be preferred as suit requirements. The values in such a lookup table will typically differ from one mat to another, and may need to be calibrated automatically or manually, possibly during manufacture or upon initial usage of the mat. It will be appreciated that impedance measurements are effected by a number of properties of the sensors such as resistance, capacitance and inductance, any of which may indicate pressure according to the configuration of the sensing mat.

### Extended System for Multiple Subjects

Other embodiments of the pressure-wound prevention system can be designed for scale and stress, aiming to monitor the accumulated pressure on a plurality of subjects. Such embodiments may include a plurality of pressure-detection mats connected to one or more drivers and control units. Power may be supplied from a plurality of sources, multiple processors may be used for calculation and analysis of the data, which may be stored in a plurality of data storage units.

Reference is now made to FIG. 1b, showing an extended pressure-wound prevention system 1000 including a plurality of pressure-wound prevention sub-systems 100a-e in communication with a common remote control center 500. The pressure-wound prevention sub-systems 100a-e may monitor various subjects in various positions for example on beds 100b, chairs 100a, 100c, 100e and wheelchairs 100d in a hospital, care home or the like and may be configured to communicate with a remote control center 500 for example at a nursing station via a data communication line. It will be appreciated that in embodiments where the pressure detection mat is configured to move such as where the subject is seated in a wheelchair or the like, wired data cables may be inappropriate and data transmission via wireless means may be preferred, for example via radio waves using protocols such as Wi-Fi, Bluetooth or the like.

Alternatively, the plurality of pressure-wound prevention sub-systems 100a-e may be located remotely from one another for example each in an individual home, and the remote control center 500 may be a manned monitoring station for the purpose. In such systems, a data communication line may be provided via a cellular network, connections to the internet or the like.

It is further noted that a single pressure-wound prevention system may include multiple pressure detection mats, for example and without limitation two mats located on a seat of a chair and on a back of a chair.

The remote control center 500 typically includes a data storage unit 560 for storing data from the sub-systems 100a-e and a display unit 570 for presenting the data as required. It will be appreciated that the control center 500 may additionally provide processing and driving functionality for controlling multiple sub-systems. Optionally each pressure-wound prevention sub-system 100a-e may have its own dedicated monitor 170 for processing, storing and displaying data locally.

Pressure Sensing Mat for Use with Pressure-Wound Prevention Systems

Embodiments of a pressure sensing mat (also referred to as a “sensing mat” or “sensor layer” herein) are disclosed. The sensing mat may be placed between a seat of a chair or a mattress of a hospital bed and the body of a seated subject. The sensing mat is typically used to monitor the pressure exerted upon the subject in a sitting or lying position. The output of the pressure sensing mat may be used to indicate the risk of pressure-wound development.

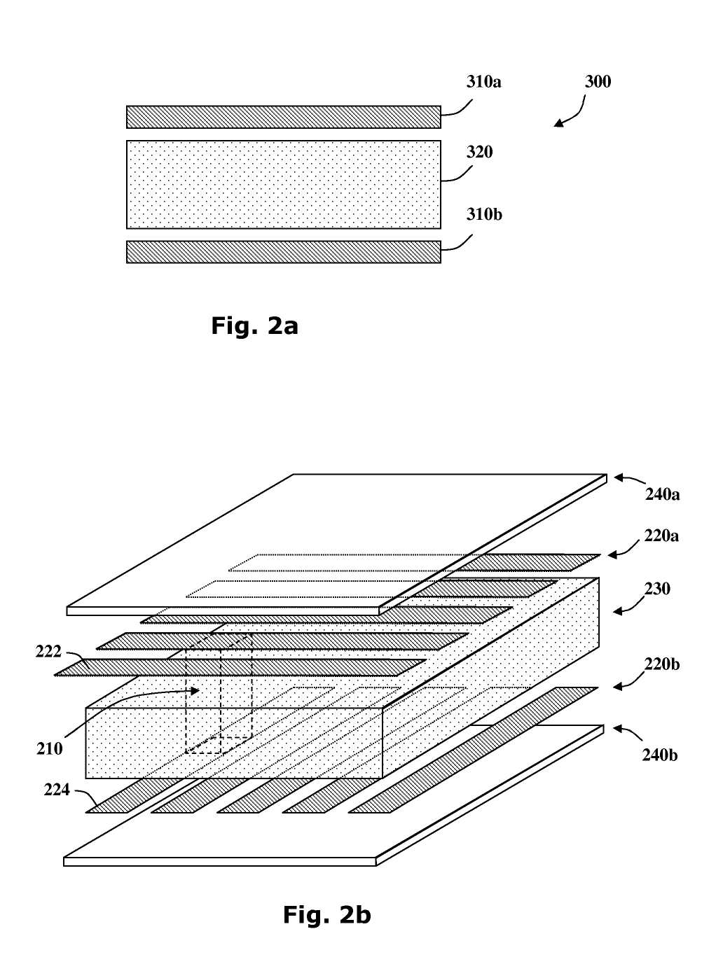

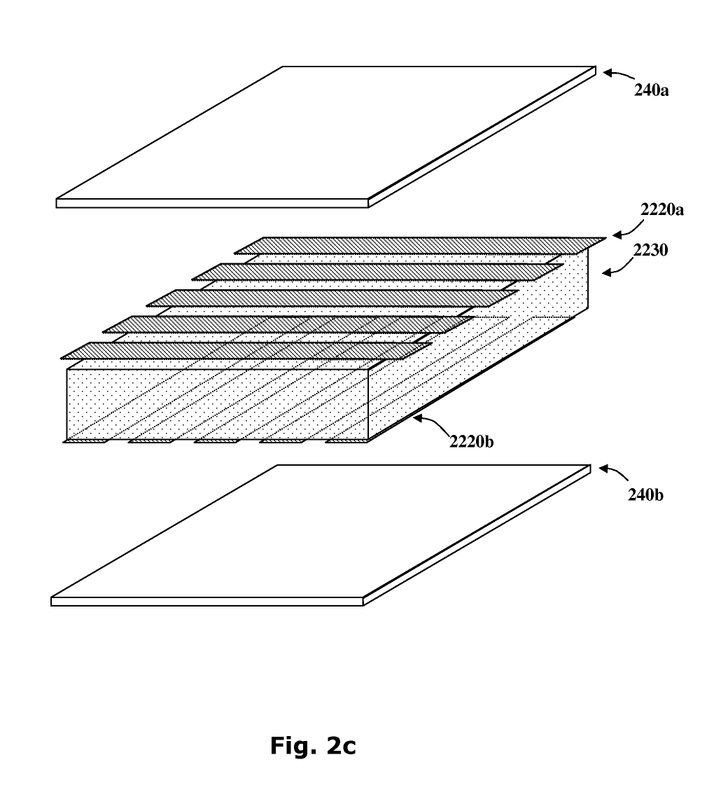

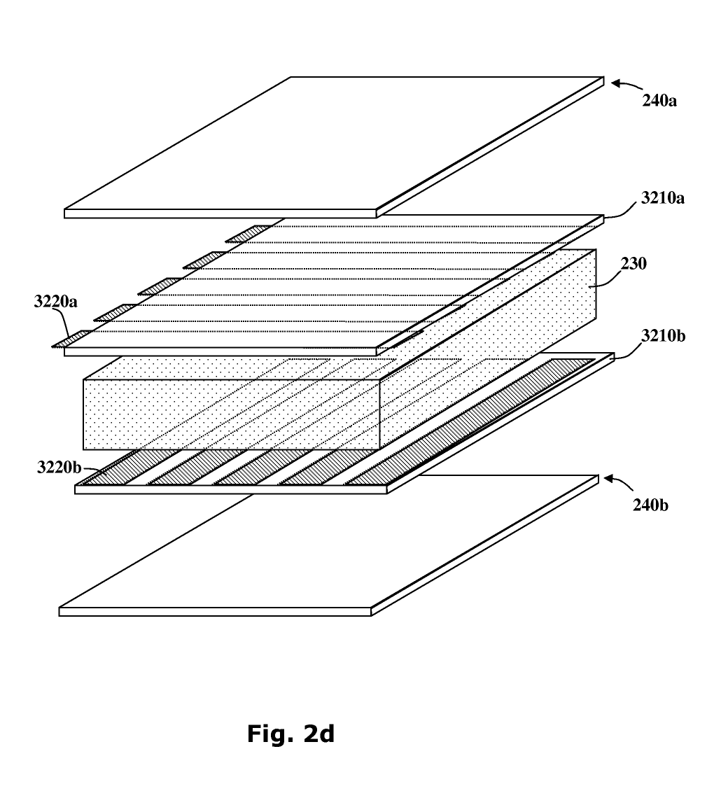

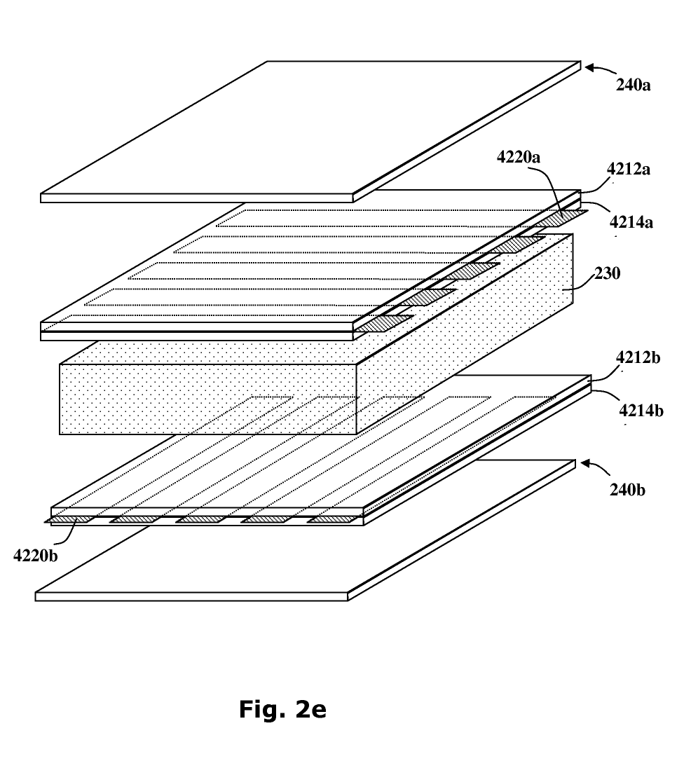

Reference is now made to FIG. 2a, showing a cross section of a basic embodiment of a single sensor 300. In this embodiment, the sensor is a capacitor comprised of two layers of conductive strips 310a, 310b and an insulating layer 320 of isolating material therebetween. Pressing anywhere on the sensor would compress the insulating layer 320 changing the distance between the conductive strips and thereby changing the capacitance of the capacitor. Although only a capacitance sensor is described, it is noted that according to other embodiments, resistance sensors may be preferred. Accordingly, the resistance of the insulating layer may be monitored as it varies according to pressure.

Reference is now made to FIG. 2b showing an isometric projection of an embodiment of a pressure-detection mat 200 comprising a plurality of sensors 210 arranged in a form of a matrix. The mat typically has two layers 220a, 220b of conductive material separated by an insulating layer 230 of isolating material. Each of the conductive layers typically consists of parallel conductive strips 222, 224 and the two conductive layers are arranged orthogonally such that in one conductive layer the strips are horizontal 222 and in the other conductive layer they are vertical 224. Each strip is wired to a control unit and is preferably operable by safe low voltage source.

A capacitance sensor is based on the capacitance between the sections of the conducting strips overlapping at each “intersection” of a vertical conductive strip with a horizontal conductive strip. These capacitance sensors are configured such that pressing anywhere on their surface changes the spacing between the two conductive layers, and consequently the capacitance of the intersection. A driving unit may selectively provide an electric potential to the vertical strip and the electrical potential may be monitored on the horizontal strip such that the capacitance sensor of the overlapping section may be determined.

It is noted that by providing an oscillating electric potential across each sensor and monitoring the alternating current produced thereby, the impedance of the intersection may be calculated and the capacitance of the intersection determined. The alternating current varies with the potential across a capacitor according to the formula:

I_{ac}=2πfCV_{ac}

where I_{ac} is the root mean squared value of the alternating current, V_{ac} is the root mean squared value of the oscillating potential across the capacitor, f is the frequency of the oscillating potential and C is the capacitance of the capacitor.

Thus where the values of V_{ac} and I_{ac} are known at a known frequency, the capacitance of a sensor may be calculated. Accordingly, where the mechanical properties of the sensor are known, the pressure applied upon the sensor may be deduced.

Preferably a capacitance sensor will retain its functionality even if it is fully pressed continuously for long periods such as or even longer than 30 days and keep its characteristics for periods over the lifetime of the sensing mat which is typically more than a year. Notably, the sensor characteristics should preferably be consistent between two separate events.

According to some embodiments, the mat may further include additional sensors configured to monitor additional factors, particularly additional factors influencing the development of bedsores, such as temperature, humidity, moisture, or the like. Such additional sensors may be configured to monitor the factors continuously or intermittently as appropriate to detect high risk combinations of factors. Such measurements may be recorded and stored in a database for further analysis.

Optionally, additional sensors may be located apart from the pressure-detection mat. For example, the mat could be integrated into a seat of a chair and a touch sensor could be integrated into a chair's back support.

In preferred embodiments of the pressure-detection mat, the materials are selected such that the conductive layers and insulating layers are flexible. The insulation material may be a compressible typically sponge-like, airy or poriferous material (e.g. foam), allowing for a significant change in density when pressure is applied to it. Materials comprising the sensing mat are typically durable enough to be resistant to normal wear-and-tear of daily use. Furthermore, the sensing mat may be configured so as not to create false pressure readings for example when the mat is folded.

The pressure-detection mat 200, or sensing-mat, may be placed underneath or otherwise integrated with other material layers 240a, 240b such as used in standard bed sheets. It will be appreciated that such additionally materials may confer further properties as may be required for a particular application. Typically, the conductive material of the sensors is wrapped by isolating, washable, water resistant, breathing cover mat, allowing minimum discomfort to the subject resting on the mat.

The sensor layer may include a Sensor Support and Connectivity Elements (SSCE) adapted to receive and electrically connect to one or more external interfaces one or more sensors of one or more sensor types. The sensor layer may also include one or more sets of sensors, wherein each set of sensors may include sensors of types such as pressure sensors, temperature sensors, humidity sensors and/or any other type of sensors known today or to be devised in the future.

An external interface according to embodiments may be integral or otherwise functionally associated with the sensor layer. An external interface according to embodiments may be adapted to receive power from an external power source and to convey the received power through the SSCE to one or more sensors connected to the sensor layer. An external interface according to embodiments may be adapted to receive through the SSCE output signals from one or more sensors connected to the sensor layer and to convey the received output signals to an external diagnostic appliance connected to the external interface. According to embodiments where the interface provides for sensor power, the interface may be either a wired or a wireless external interface such as an inductive power coupling interface. According to embodiments where the interface provides for sensor signal output, the interface may be either a wired or a wireless external interface such as a Wi-Fi interface, a Bluetooth interface and the like. An external interface may include an interface panel, and the interface or interface panel may include elements such as: (1) wired connectors for interfacing with an external diagnostic appliance, (2) wired connectors for interfacing to an external power source, (3) wireless communication circuits for communicating with an external diagnostic appliance, (4) inductive circuits for interfacing to an external power source, and/or (5) any combination of the aforementioned elements.

Sensor Support and Connectivity Elements (SSCE) according to embodiments may include a set of electrically conductive wires or strips, wherein the conductive wires or strips may include metallic conductors or may include non-metallic electrically conductive materials. According to some embodiments, one or more sensors, such as pressure sensors may be integral with the electrically conductive wires or strips. Applicant's co-pending Patent Cooperation Treaty applications, PCT/IL2010/000294 and PCT/IB2011/051016, teach such a sensor to conductive wires/strips integration, and both applications are hereby incorporated by reference into the present application in their entirety. According to embodiments where a sensor is not integrated with the electrically conductive wires or strips, some of the electrically conductive wires or strips may include or be electrically connected to one or more sensor mounting structures, connectors or sockets to which a sensor (e.g. pressure, temperature, humidity, etc.) may be connected.

The electrically conductive wires or strips may form a grid across the sensor layer or across a portion of the sensor layer. Some or all of the conductive wires or strips may terminate at an external interface. According to some embodiments, all of the terminating conductive wires or strips may terminate at the same external interface. According to further embodiments, a first set of the terminating conductive wires or strips may terminate at a first external interface and a second set of the terminating conductive wires or strips may terminate at a second external interface. According to some embodiments, all of the conductive wires or strips may include or be connected to sensors of the same sensor type. According to further embodiments, a first set of the conductive wires or strips may include or be connected to sensors of a first sensor type and a second set of the conductive wires or strips may include or be connected to sensors of a second sensor type.

With reference now to FIGS. 2c-e showing exploded views of various embodiments of the pressure-detection mat, the conductive layers 220 (FIG. 2b) may be supported by various substrates. For example FIG. 2c shows two conductive layers 2220a, 2220b adhered directly to the insulating layer 230. Alternatively, as shown in FIG. 2d, conductive layers 3220a, 3220b may be supported by separate substrates 3210a, 3210b, such as of Thermoplastic Polyurethane (TPU) for example, the insulating layer 230 being sandwiched therebetween. In still another embodiment, as shown in FIG. 2e, the conductive layers 4220a, 4220b may themselves each be sandwiched between two substrates 4212a, 4214a, 4212b, 4214b respectively.

It will be appreciated that in order to get a stable reading of impedance values from a row of sensors, it is preferable that little or no movement be made by the subject during the taking of readings from the sensors. Accordingly, according to certain embodiments the response time of the sensors and the time taken for readings should be small possibly of the order of tens or hundreds of milliseconds, during which movement of the subject is generally insignificant although other response times may be required as appropriate. It is particularly noted that in applications where the subject is largely immobile, it may be advantageous to use longer reading times.

The pressure-detection mat, or sensing-mat is typically placed on surfaces such as a mattress of a hospital bed, a long term care facility bed, a home bed, a seat or a back of a chair, a couch, a wheelchair, or the like. Embodiments of this system can detect the pressure points formed between a subject resting on one or more pressure-detection mats and the surface upon which the mats rest. Surfaces may be parts of chairs, stools, sofas, wheelchairs, rocking chairs, chaise longue, banquets, bean bags, ottomans, benches and poufs. Pressure mapping data per subject may be aggregated over time in one or more data storage units.

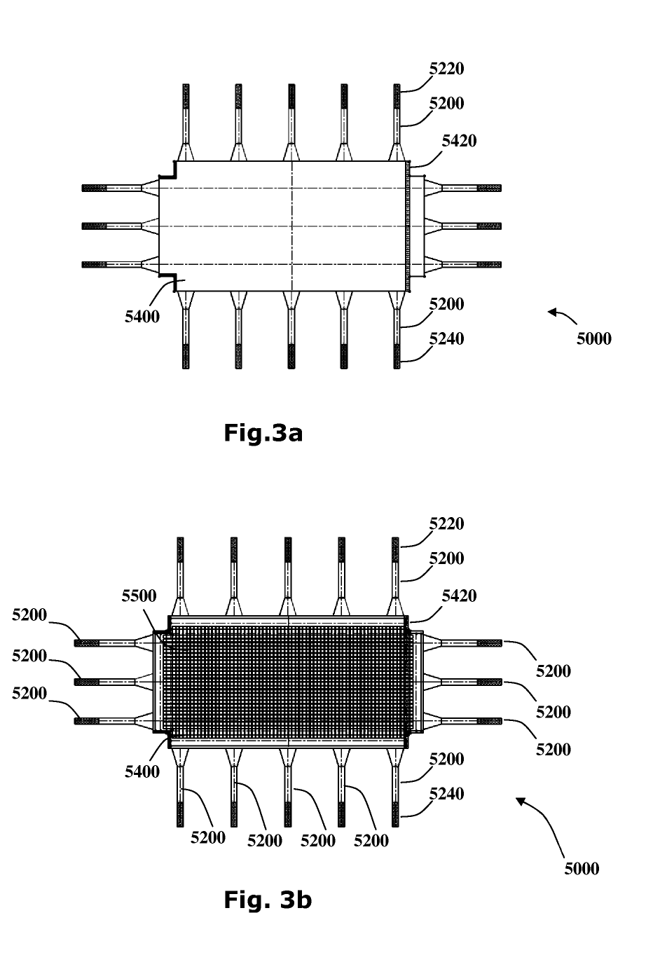

With reference to FIGS. 3a and 3b, a top view and section through respectively are shown of a further embodiment of a pressure detection mat 5000. The pressure detection mat 5000 includes a sensor matrix 5500, such as described hereinabove, housed within a cover mat 5400 and which may be sealed by a zipper 5420 as required.

The pressure detection mat 5000 may be attached to a surface in such a way that prevents movement of the mat relative to the surface. A feature of the embodiment of the mat 5000 is that the cover mat 5500 may include a coupling mechanism for securing the mat to a seat or a back of a mattress, a bed, a chair, a bench, a sofa, a wheelchair or the like. The coupling mechanism may include for example at least one strap 5200 having an attachment means 5240 configured to secure the straps 5200 to the seat or to each other such that the pressure detection mat is held securely. This may be useful to prevent folding, wrinkling or other movement of the detection mat which may contribute to the creation of shear forces which are known to encourage the formation of external pressure sores. Suitable attachment means include for example, hook-and-eye materials such as Velcro®, buckles, adhesives, buttons, laces or such like as suit requirements.

### Sensor Integrated Resting Body Support

Embodiments disclosed herein include methods, circuits, apparatuses and assemblies for providing a mattress or body portion support cushion (referred to collectively herein as “resting body support”) with a sensing mat having an array of sensors. The resting body support may be any item providing support for a body or portion thereof, including but not limited to a mattress, a seat, a cushion, a backrest, a handrail, a footrest, and the like. According to some embodiments, there may be provided a sensor integrated resting body support, i.e., a sensing mat integrated with a resting body support. For example, the sensing mat may be embedding within a resting body support or provided within an encasement of a resting body support. In the context of being integrated with a resting body support, the sensing mat may be referred to herein as a “sensor layer”.

The one or more sensors of the sensor layer may be of one or more sensor types including pressure sensors, temperature sensors, humidity sensors and/or any other kind of sensors known today or to be devised in the future.

According to some embodiments, the sensor layer may include one or more sensors of a single or of multiple sensor types. Some or all of the sensors may be adapted to sense parameters relevant to the wellbeing of a person being supported by a resting body support including the sensor layer. Parameters sensed by sensors of the sensor layer may include: (1) pressure applied by a given portion, limb or extremity of the person being supported, (2) temperature of the given portion, limb or extremity of the person being supported, (3) humidity of the given portion, limb or extremity of the person being supported, etc.

According to some embodiments, all of the sensors of the sensor layer may be electrically connected to the Sensor Support and Connectivity Elements (SSCE), as described herein, of the sensor layer. At least some of the sensors of the sensor layer may be arranged in a grid pattern, for example at least partially mirroring a grid pattern of the SSCE conductors. According to further embodiments, only a portion of the sensors of the sensor layer may be electrically connected to the SSCE of the sensor layer.

Some sensors of the sensor layer may be removable sensors. Some removable sensors may be adapted to electrically connect with a connector or socket of the SSCE. Other removable sensors may be adapted for placement into a receptacle of a sensor layer according to embodiments, and may be adapted to output sensor signals through proprietary wiring or wirelessly. Removable sensors according to embodiments may be powered either by an internal battery, through wiring, or wirelessly using inductive power coupling.

A sensor layer according to embodiments may be produced as a discrete layer separate from a resting body support or from a resting body support encasement with which the sensor layer is used. According to such embodiments, the sensor layer may be produced by implanting or embedding an SSCE along with associated sensors into either a protective coating or into a sheet-like material. The discrete sensor layer may subsequently be inserted into or integrated with a resting body support such as a mattress or cushion. The discrete sensor layer may subsequently be inserted into or integrated with a resting body support encasement such as a mattress encasement or cushion encasement. An external interface according to some embodiments may be connected to a discrete sensor layer prior to insertion or integration with the resting body support or encasement. An external interface according to further embodiments may be connected to a discrete sensor layer after insertion or integration with the resting body support or encasement.

Alternatively, a sensor layer according to other embodiments may be produced concurrently with a resting body support or resting body support encasement. According to such embodiments, production of a sensor layer, during production of the resting body support or resting body support encasement, may include depositing the SSCE on an inner layer of the resting body support or resting body support encasement, wherein the deposition is done either directly or through an intermediate material. If sensors of the sensor layer are not integral with the electrically conductive wires or strips of the SSCE, sensors may be electrically connected to the SSCE. According to some embodiments, sensors may be placed prior to deposition of the SSCE, and SSCE deposition may include electrical interconnection between the sensor and the electrically conductive wires or strips of the SSCE. One or more external interfaces according to such embodiments may be electrically connected to the sensor layer of the resting body support or encasement either prior to or after the support or encasement are completed and/or sealed.

Some embodiments include a resting body support such as a mattress, a cushion, a seat cushion, a wheelchair cushion, or the like. The resting body support may be integral with a sensor layer, being a sensor integrated resting body support, which may be connected with an external interface. A resting body support according to embodiments may provide sensor signals indicative of one or more parameters relevant to the wellbeing of a person using the support. An external diagnostic device may receive the sensor signals through the external interface. The resting body support may be sealed, for example hermetically sealed. A resting body support according to embodiments may include one or more fastening elements, such as buckles or snaps, to which a protective sheet may be fastened.

Some embodiments of the present invention include a resting body support encasement for encasing a resting body support such a mattress, a cushion, a seat cushion, a wheelchair cushion, or the like. The encasement may be integral with a sensor layer and optionally with an external interface. A resting body support encasement according to embodiments may provide sensor signals indicative of one or more parameters relevant to the wellbeing of a person using the support. An external diagnostic device may receive the sensor signals through the external interface. The resting body support encasement may be sealed, for example hermetically sealed. A resting body support encasement according to some embodiments may include one or more fastening elements, such as buckles or snaps, to which a protective sheet may be fastened.

A resting body support encasement according to some embodiments may include one or more adjustable straps, connected to either an inner or outer surface of the encasement, for adjusting an effective size of the encasement. The adjustable straps and resulting effective size adjustment functionality may enable the use of a single resting body support encasement model with a range of resting body supports having a range of sizes and/or dimensions. The resting body support encasement may also include one or more pockets or other receptacles, either on an inner or outer surface of the encasement, for receiving one or more removable sensors of one or more different sensor types.

According to some embodiments, such as those where the sensor layer is discrete, the sensor layer may be attachable and detachable from a surface of the encasement. The discrete sensor layer may be attachable and detachable from an inner surface of the encasement, and the encasement may be disposable.

Embodiments disclosed herein include methods, circuits, devices, apparatuses, encasements and systems for providing a pressure sensor array which is either integral with a mattress or is integral with a mattress encasement (i.e. A Mattress Sensor Layer). According to some embodiments, there may be provided a pressure sensor lined mattress, wherein an array of pressure sensors (e.g. capacitive or piezo-material-based sensors) may be embedded: (1) within the mattress body or (2) within a portion of (e.g. side) an encasement of the mattress. Some or all of the sensor array elements (e.g. sensors) may be electrically connected, via a set of electrically conductive wires, to an external interface panel with connectors for interconnection to an external appliance. According to embodiments where the sensor array is within a mattress body, the panel and connectors may be integral with the mattress body. According to embodiments where the sensor array is within a mattress encasement, the panel and connectors may be integral with the mattress encasement.

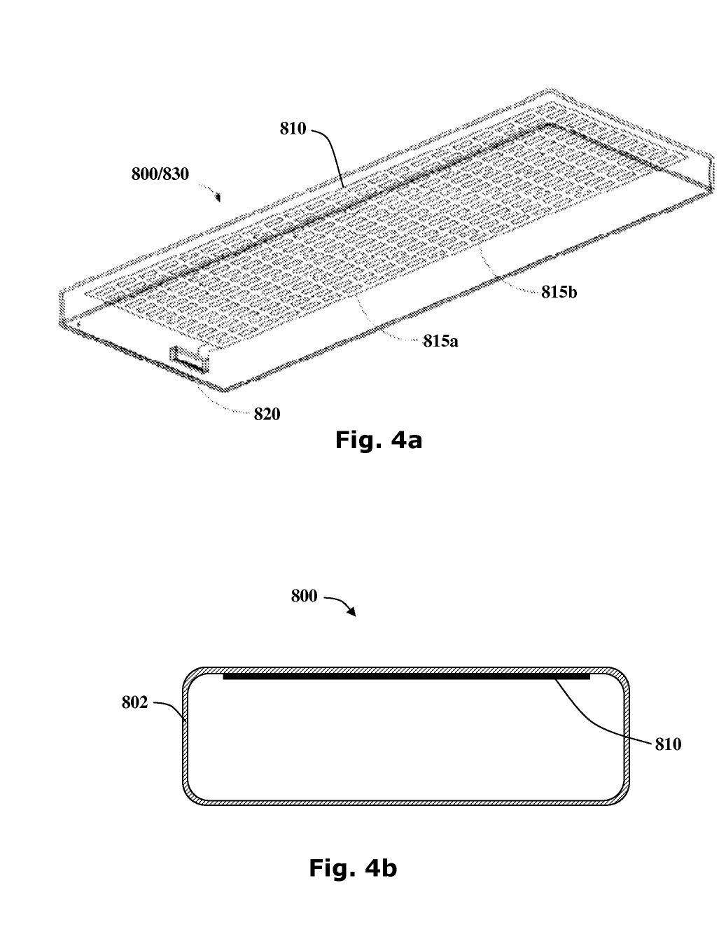

Turning now to FIG. 4a, there is shown a top perspective view of an exemplary sensor layer 810, including sensors 815a and 815h such as pressure sensors, according to embodiments. The sensors 815 are arranged in a grid and are electrically connected to an external interface 820. The sensor layer may be integrated into, e.g., a mattress 800 or a mattress encasement 830.

FIG. 4b shows a cross-sectional view of an exemplary mattress 800 with an integrated sensor layer 810 according to embodiments, where the sensor layer 810 is integrated in a portion of the mattress near the surface of the mattress, underneath an outer portion 802.

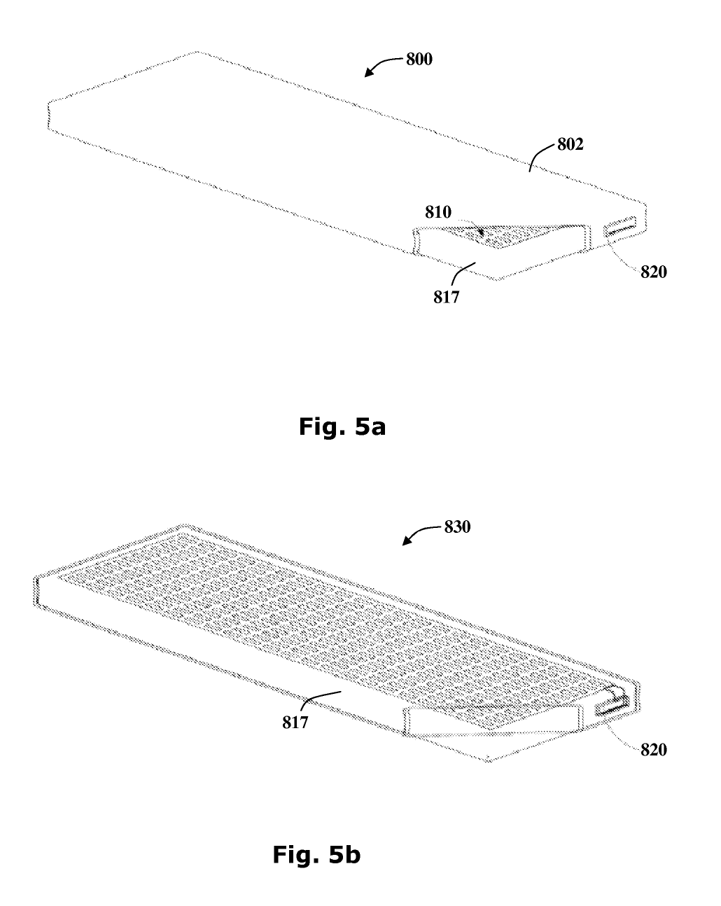

Turning now to FIG. 5a, there is shown a perspective view with cutaway of an exemplary sensor integrated mattress 800, with an integrated sensor layer 810 according to embodiments. FIG. 5a includes a cutaway on the bottom left corner of the mattress outer portion 802, thereby revealing the integrated sensor layer 810 and the cushioning material 817. Also shown in FIG. 5a is the external interface 820, which may be configured to be situated on the outer surface of the mattress 800.

Turning now to FIG. 5b, there is shown a perspective view with cutaway of an exemplary sensor integrated mattress encasement 830 having an integrated sensor layer 810 according to embodiments with an encasement substrate 835. The top of the mattress encasement 830 is also shown as being transparent in order to provide an understanding of the coverage area of the sensor layer 810 as well as the interconnection of the sensor layer 810 with the external interface 820 panel.

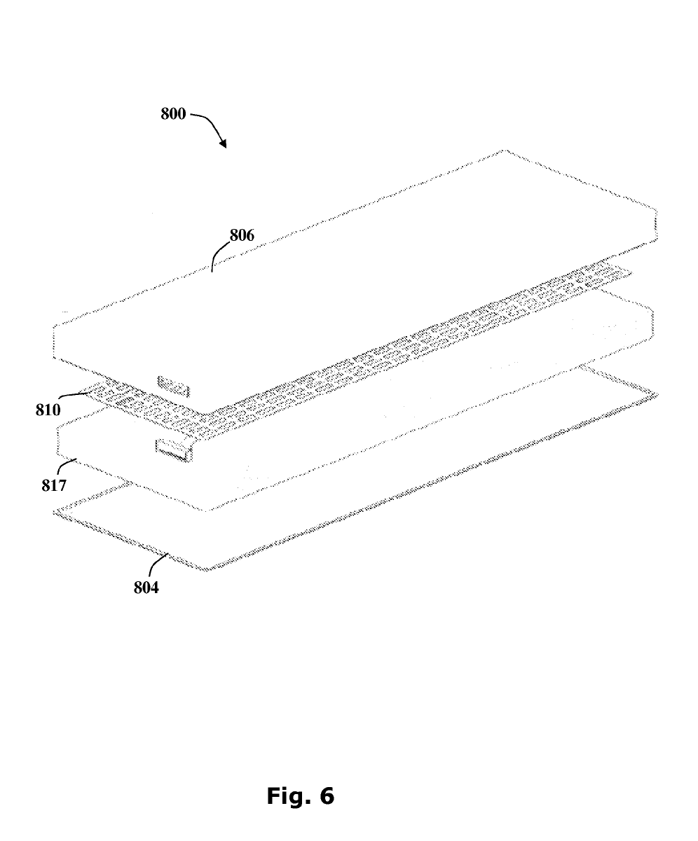

Turning now to FIG. 6, there are shown elements and steps of constructing an exemplary sensor integrated mattress 800 with an integrated sensor layer 810 according to embodiments. Onto the base layer 804 may be placed the cushioning material 817. The sensor layer 810 may be affixed in-between a top surface of the cushioning material 817 and an inside surface of a covering layer 806. It should be understood by one of skill in the art that numerous variations of this arrangement and sequence are within the spirit and scope of the present invention.

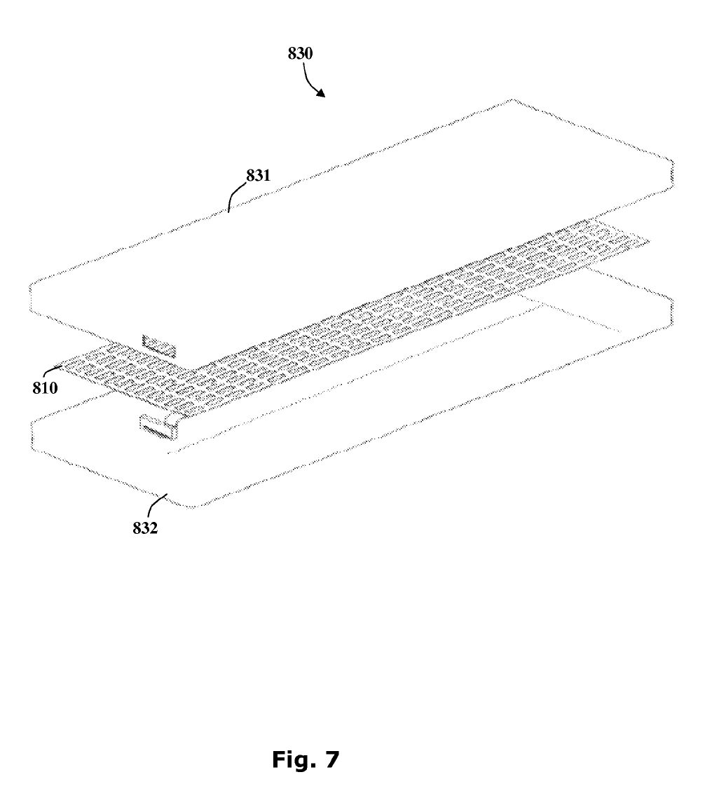

Turning now to FIG. 7, there are shown elements and steps related to the construction of an exemplary mattress encasement 830 with an integrated sensor layer 810 according to embodiments. The mattress encasement 830 may comprise a top portion 831 and a bottom portion 832. The sensor layer 810 may be affixed to a bottom/inner surface of the mattress encasement top portion 831, either prior or concurrent to the mattress encasement top portion 831 being bound to the mattress encasement bottom portion 832. The mattress encasement bottom portion 832 may include a zipper or other temporary sealing means, symbolized with dashed lines, for facilitating the removable application of the encasement onto a mattress (not shown). It should be understood by one of skill in the art that numerous variations of this arrangement and sequence are within the spirit and scope of the present invention. With further reference to FIGS. 4-7, it will be appreciated that the mattress 800 may be any resting body support (e.g., a mattress, a seat, a cushion, a backrest, a handrail, a footrest, and the like), and that the mattress encasement 830 may be an encasement for any resting body support.

### Mattress Adjustment System

In still another embodiment, the sensor sheet may be used in a combination with an active mattress optionally having a matching grid of cells, as a mattress adjustment system. In this embodiment, when pressure exceeds a given threshold, neighboring mattress cells will inflate or deflate to redistribute the pressure. It will be appreciated that such an active solution may reduce the necessity to turn or reposition the patient. Accordingly, in certain embodiments, pressure monitoring and relief may be completely automated.

In the mattress adjustment system, the sensor sheet, being a pressure-detection mat having a plurality of sensors and at least one processor, may be used in combination with an active mattress having a plurality of cells, each cell being individually inflatable. Each cell may be connected to an inflation control unit. The level of inflation of each cell may be individually controllable by the control unit. Alternatively or additionally, as required, pluralities of cells may be grouped into cell clusters and jointly controllable by a common inflation control unit.

Cell inflation refers to any change in the physical characteristic of the cell that increases the pressure applied to the subject at the location of the adjusted cell, and cell deflation refers to any change in the physical characteristic of the cell that decreases the pressure applied to the subject at the location of the adjusted cell. The physical characteristic of the cell may be, without limitation, pressure, firmness, height, size and the like. Cell inflation and deflation may be mediated by, e.g., air, liquid, mechanical actuators, and other mechanisms that can adjust the pressure applied to the subject situated on the active mattress. In a particular embodiment, cell inflation may be mediated by air.

The sensor sheet, being a pressure-detection mat having a plurality of sensors and at least one processor, which may be used in combination with an active mattress having a plurality of cells, each cell being individually inflatable. Each cell may be connected to an inflation control unit. The level of inflation of each cell may be individually controllable by an inflation control unit. Alternatively or additionally, as required, pluralities of cells may be grouped into cell clusters and jointly controllable by a common inflation control unit.

The active mattress may be operable to be retrofitted onto the pressure-detection mat 200. Alternatively, the active mattress may be integrated with the pressure-detection mat.

The level and pattern of inflation may be adjusted based on the pressure exerted by a patient's body that is on the active mattress. As such, the inflation control unit may be connected to the pressure detection mat, e.g., to the processor configured to determine pressure at the sensors of said pressure detection mat.

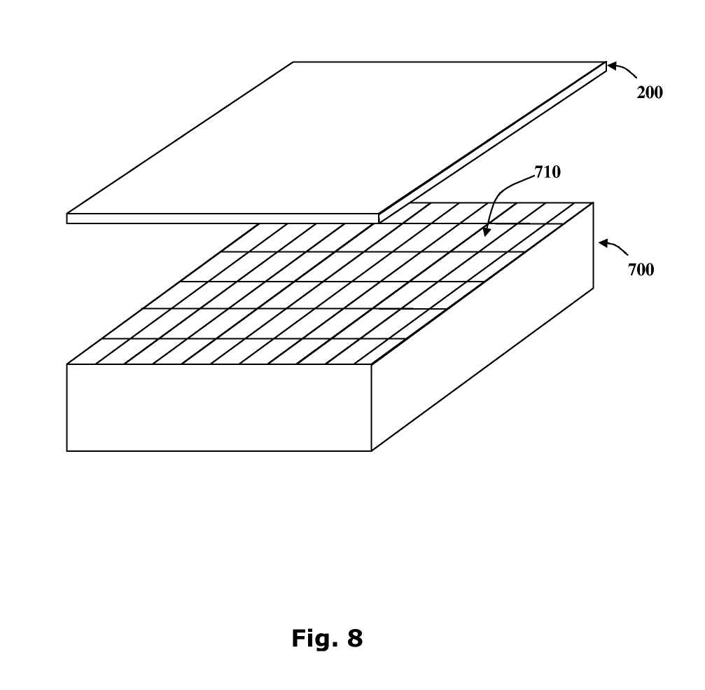

As shown in FIG. 8, the active mattress 700 may be placed under the pressure detection mat 200. Alternatively, if required, the active mattress 700 may be placed on top of the pressure-detection mat 200.

The processor may be further configured to detect a risk locus, which may be defined as a location on the pressure detection mat 200 corresponding to one or more sensors 210 detecting pressure values exceeding a predefined threshold for a predefined duration. The predefined duration may be instantaneous, or an extended period of time, e.g., seconds, minutes, hours and the like. Further, the predefined duration may be dependent on the predefined threshold pressure, wherein the region corresponding to the sensor detecting a higher predefined threshold pressure requires a shorter predefined duration for the area corresponding to the sensor to be detected as a risk locus.

The active mattress 700 may be configured such that when the pressure-detection mat 200 detects a risk locus, one or more cells 710 at or surrounding the risk locus adjusts the level of inflation, e.g., inflate or deflate, in order to redistribute the pressure applied to the sensors at and/or around the risk locus. The redistribution may include reducing the pressure at the risk locus. Alternatively or in addition, the redistribution may include increasing or reducing the pressure of a peripheral area neighboring or surrounding the risk locus. It will be appreciated that such an active solution may reduce the necessity to turn or reposition the patient. Accordingly, in certain embodiments, pressure monitoring and inflation adjustment may be completely automated. The cycle of risk locus detection by the pressure-detection mat 200 and the cell inflation adjustment in the inflatable mat 700 may be repeated. This cycle may be repeated a predetermined number of times or a predetermined amount of time. Alternatively, the cycle may be started and stopped according to start/stop instructions entered manually by the subject or a user through, e.g., a user interface.

### Coordinated Inflation/Deflation of Multiple Cells.

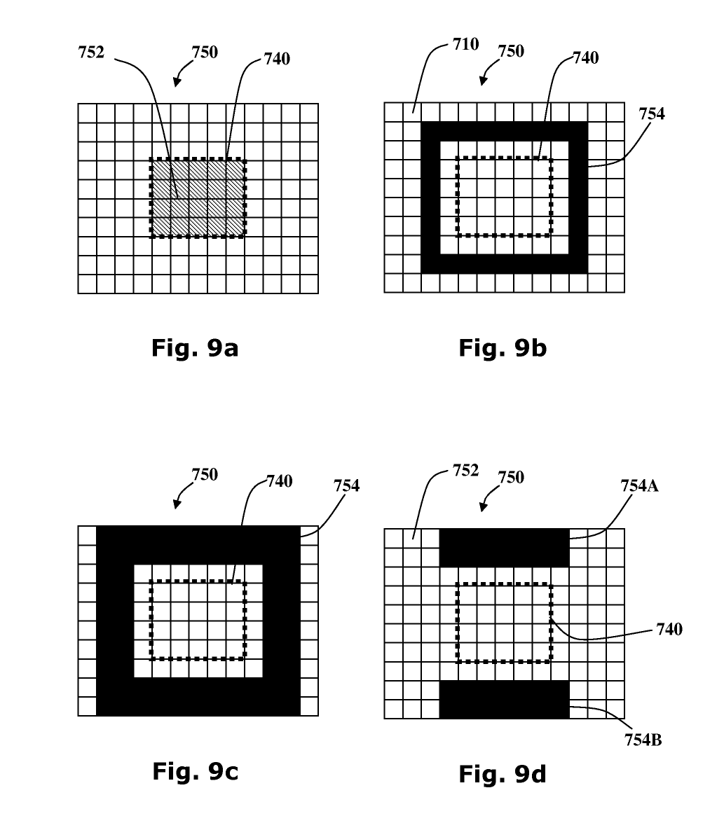

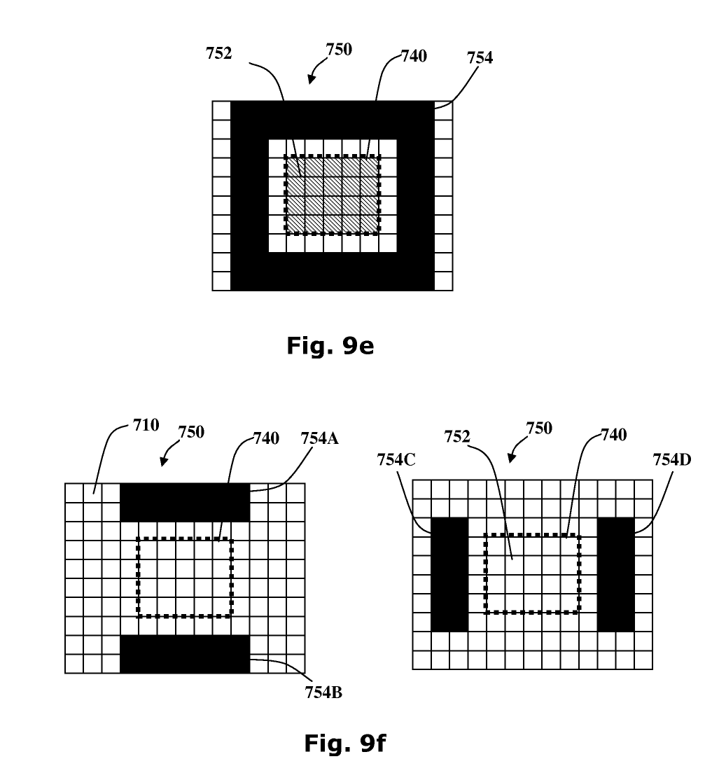

With references to FIGS. 9a-f, the active mattress 750 may be configured such that, upon detection of a risk locus 740, the active mattress 750 adjusts the level of inflation of one or more of the cells 710, e.g. into a pattern of inflation and/or deflation of one or more cells 710. The cell inflation level may be adjusted such that the force present on the patient's body at said risk locus 740 is reduced. More specifically, the cell inflation may be adjusted such that one or a combination of the following pressure redistribution targets are achieved:
The pressure values measured by the sensors corresponding to the risk locus are lowered such that the risk locus is no longer designated as a risk locus.
The pressure values at the risk locus are lowered to zero (i.e., no pressure).
Other loci outside the risk locus do not become designated as a risk locus as a result of the cell inflation adjustment.

The cycle of risk locus detection by the pressure-detection mat and the cell inflation adjustment in the inflatable mat may be repeated to continually evaluate the effectiveness of the previous round of cell inflation adjustment in achieving one or more of the pressure redistribution targets, and to further adjust cell inflation if needed, and/or to continue adjusting the pressure redistribution as the subject's position changes.

Some exemplary inflation adjustment patterns are presented hereinbelow:
With reference to FIG. 9a, the detection of a risk locus 740 may trigger the mattress 750 to deflate the cells 752 corresponding to the location of the risk locus 740. The deflated cells 752 are shaded. In addition, a peripheral area of cells 752 neighboring the risk locus may be deflated as well.
With reference to FIGS. 9b-d, the detection of a risk locus 740 may trigger the mattress to 750 to inflate the cells 754 surrounding the location of the risk locus. The cells 754 selected to be inflated may fully surround the risk locus 740, as shown in FIG. 9b. The thickness of the area of the inflated cells 754 may be thin, as shown in FIG. 9b, or thicker, as shown in FIG. 9c. Alternatively, as shown in FIG. 9d, the active mattress 750 may be inflate cells 754A, 754B on opposing sides of a risk locus 740, without the inflated cells 720 fully surrounding the risk locus 740. Such an arrangement may serve to, e.g., alleviate the pressure on the risk locus 740 while enabling better blood flow or air flow to the risk locus 740, compared to a fully-surrounding set of inflated cells 754. The inflated cells 754 are shown as black.
With reference to FIG. 9e, the detection of a risk locus 740 may trigger the mattress 750 to deflate the cells 752 corresponding to the location of the risk locus as well as inflate the cells 752 surrounding the location of the risk locus. The inflated cells 754 are shown as black and the deflated cells 752 are shaded in FIG. 9e.
In addition, the active mattress may be configured to change the pattern of inflation/deflation of the cells over time. Such an arrangement may serve to, e.g., to reduce the duration of elevated pressure at the areas surrounding the risk locus and extend the period of time that the subject can stay in a given posture on the active mattress 750. For example, the inflation/deflation pattern may cycle through two or more of the patterns described hereinabove. FIG. 9f shows a particular example of a cycling of two patterns: (1) two areas of cells 754A, 754B on either side of the risk locus 740 being inflated along the vertical axis; and (2) two areas of cells 754C, 754D on either side of the risk locus 740 being inflated along the horizontal axis.

### Active Mattress Embodiments

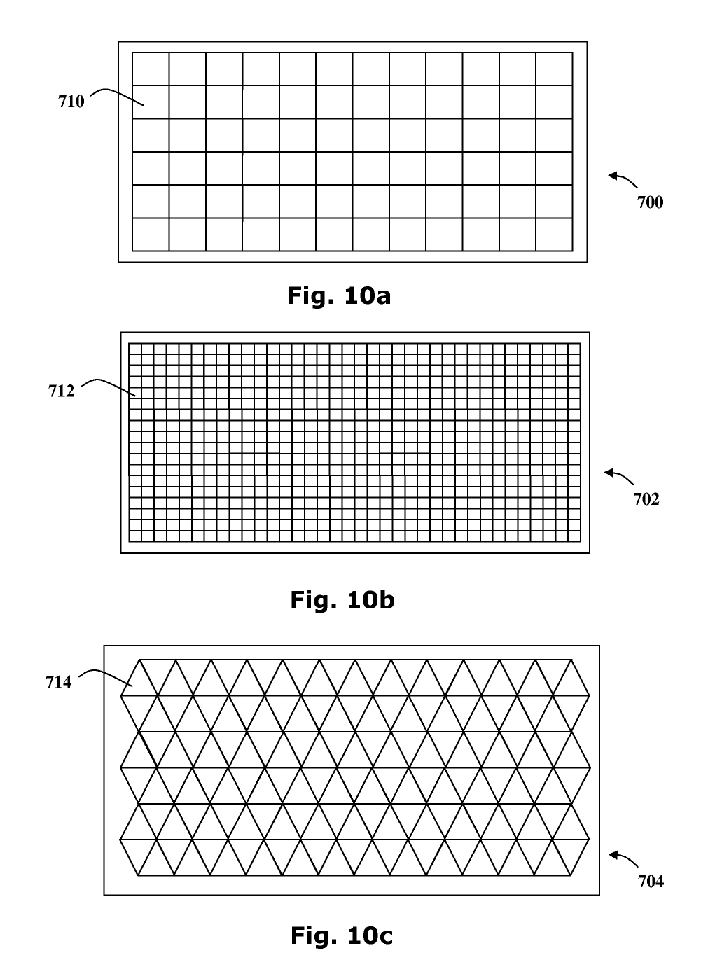

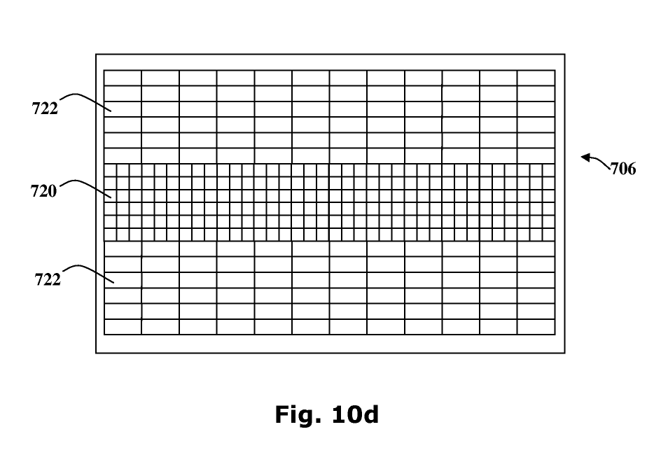

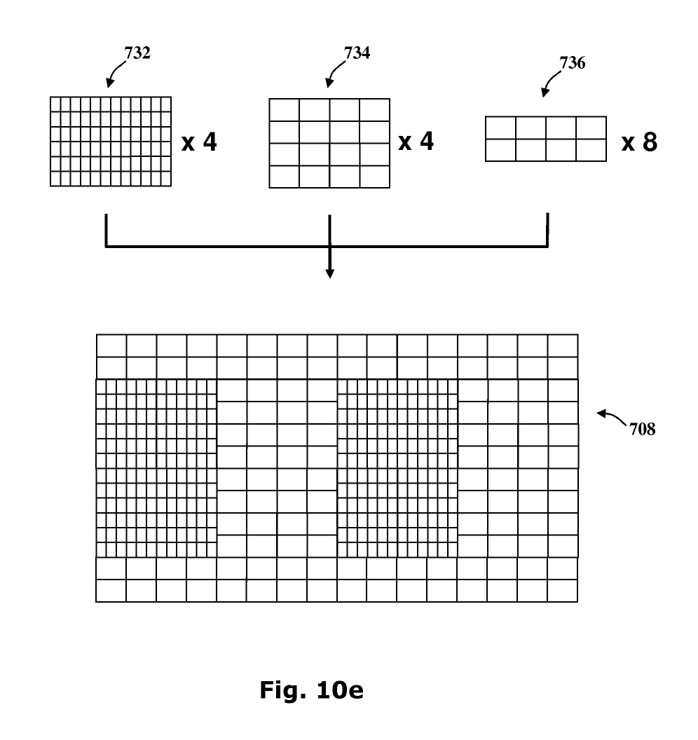

Reference is now made for FIGS. 10a-d. Each cell 710 on the active mattress 700 may be identical in dimensions, thickness, baseline firmness, and other parameters. Further, the cells 710 may be arranged in a matrix that corresponds with the matrix of sensors on the pressure-detection mat 200. The sensors 210 of the pressure detection mat 200 may be arranged in a grid, and the cells 710 active mattress 710 may be arranged in a matching grid. For example, each cell 710 may cover an area corresponding to the area covered by, e.g., one, two, three, four or more sensors. Alternatively, the active mattress 700 may include cells 710 of various dimensions and shapes.

Each cell 710 may be shaped as appropriate. For example, an active mattress 700 having a regular matrix of identical cells 710 may be, e.g., rectangular, triangular, hexagonal and the like. FIG. 10a shows an active mattress 700 having a regular matrix of rectangular cells 710. FIG. 10b shows and alternative active mattress 702 having a regular matrix of rectangular small cells 712. FIG. 10c shows an alternative active mattress 704 having a regular matrix of triangular cells 714. As a further alternative, the active mattress 700 may include cells 710 of various shapes, as appropriate.

Referring now to FIG. 10d, in certain embodiments, cells 710 in the active mattress 700 may be patterned such that the areas most likely to have (or designed to support) a body part vulnerable to bedsore formation positioned thereupon have smaller cells 710, enabling finer spatial control of the force exerted on the vulnerable body parts by the active mattress 700. Such vulnerable body parts are typically those with a bony protrusion, including but not limited to the sacrum, coccyx, heels, hips, elbows, knees, ankles or the back of the cranium. FIG. 10d shows, for example, an active mattress 706 having a row of smaller cells 720 down the middle of the mattress where a subject is more likely to be present, which are flanked on both sides by rows of larger cells 722.

### Modular Design—the Pattern of Big/Small Cells can be Rearranged.

Referring now to FIG. 10e, multiple active mattresses 700 (or the multiple units of the integrated active mattress 700/pressure-detection mat 200 combination) may be operable to be attached and functionally connected together. As such, multiple active mattresses 700 may be combined to form one larger active mattress 700. Further, the ability to connect multiple active mattresses 700 enables a modular design. For example, as shown in FIG. 10e, small active mattresses 732, 734, 736 of different sizes and having different cell sizes may be connected to form a larger active mattress 708, customized for a given patient or class of patients in which the cells are patterned such that the areas most likely to have a body part vulnerable to bedsore formation have smaller cells. The active mattress 708 of FIG. 10e, for example, may be best suited for patients with a vulnerability to bedsore formation on the head and hip areas.

The number of pressure detection mats may vary according to need. Pressure detection mats are typically integrated to areas of a bed or a sitting apparatus which are designed to hold body parts that are prone to develop pressure-wounds. For example and without limitation, areas of a sitting apparatus may be a chair or a sofa's seats, backs, arms, back rails, restraints, leg rests or the like, which may support body parts such as but not limited to the neck, lower back, ankles or heels.

### Further Embodiments of the Sensing Mat

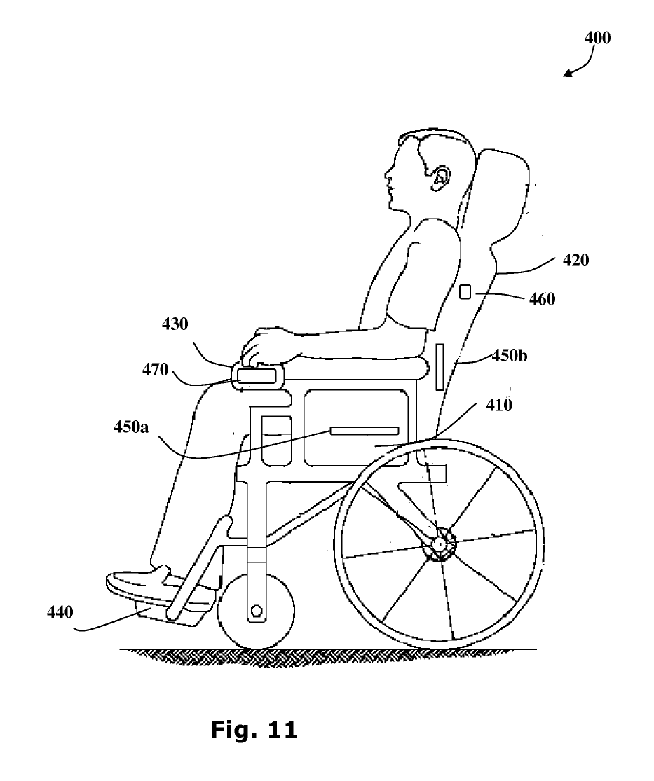

It will be appreciated that multiple embodiments of the pressure-detection mat may be located on a common sitting apparatus. Multiple embodiments of the pressure detection mat on a common sitting apparatus are demonstrated in FIG. 11, showing an embodiment of a pressure detection system integrated into a wheelchair. Embodiments of the pressure detection mats may be integrated, for example and without limitation, into the seat 410, the back 420, the arm rests 430 and the foot rests 440.

Referring back to FIG. 1a, the pressure-wound prevention system may include a power source 110 or be connected to an external power source for example and without limitation via an electric cord. In case the pressure-wound prevention system is coupled with a mobile sitting apparatus, it is important that the power source be chargeable. In electric wheelchairs, the existing battery incorporated within the electric wheelchair can further be used to supply power to the pressure-wound prevention system. In other embodiments of a sitting apparatus such as a mechanical wheelchair, a dedicated power source may be used to provide electricity to the pressure-wound prevention system. Various power sources may be usefully integrated into the system as required such as amongst others electrochemical cells, fuel cells, capacitors, solar cells, inductive power supplies, power harvesters and the like.

In various embodiments, the pressure-detection mat may further include additional sensors which can be used to detect additional environmental parameters such as temperature, humidity, ambient pressure and the like. More embodiments may further include sensors which are not integrated into the mat, aiming to detect parameters other than pressure, for example and without limitation sensors configured to detect contact between a subject and a platform. Such contact detection sensors may be placed for example and without limitation in the top rail and the cross rail of a back of a chair. Detachment of a subject from the back of the chair may result in the subject falling off the chair altogether. Therefore, information obtained from contact sensors placed in the locations mentioned earlier can be processed and used in determining whether there's danger that a subject is about to fall.

FIG. 11 illustrates how different components of a pressure-wound prevention system may be integrated into a wheelchair. The wheelchair includes a seat 410, a back 420, hand rails 430 and foot rests 440. An integrated power-source and driving unit 460 is located beneath the seat, providing power to sensing mat 450a integrated to the wheelchair seat, to a second sensing mat 450b integrated with the lower part of the back of the wheelchair, and to a touch sensor 460 located on the top rail of the wheelchair. The processing unit and the storage unit (no shown) may also located beneath the seat. A display screen 470 may be integrated into the hand rails.

In various embodiments, the data storage unit is mobile, and can be moved along with the patient from one sitting apparatus to another. Mobility of the storage unit helps preserve the pressure history of a patient as he is being moved from one room to another, or from one position to another, for example and without limitation from a hospital chair to a hospital bed or from a wheelchair to a car seat. This feature is particularly useful because moving a subject from a lying position to a sitting position does not necessarily relieve accumulated pressure applied upon all body parts.

It is a further aim of the system and method described herein to enable storage of data collected from multiple subjects in a variety of situations and a plurality of locations. Data storage is typically aggregated in one or more database units. Data storage may serve for statistics collection regarding a particular mat or line of mats, comparison of care settings according to patients' groups (for instance diabetic patients), or for the creation of a research tool designed to provide practical recommendations for turning schedules and standard of care.

### Data Analysis and Display

A software application is typically used to retrieve data from at least one data storage unit, analyze it for different purposes, and display the analysis results in various formats to a user. The software application may include features such as, but not limited to:
Calculating and presenting pressure detected by each sensor on a pressure-detection mat;
Calculating shear forces pressures by comparing relative pressures detected by adjacent pixels;
Calculating and presenting the accumulated pressure over time detected by each sensor on a pressure-detection mat;
Calculating and presenting data such as temperature or moisture build-up over time;
Calculating and alerting a caretaker at a monitoring station when patients need to be moved in order to prevent the creation of pressure-wounds;
Alarming when a pressure beyond a predefined threshold and a predefined duration is reached.
Calculating, presenting and alarming about different mat parameters, such as but not limited to wireless transmission malfunction, electricity disconnection, or the like.
Calibrating pressure-detection sensors comprising the pressure-detection mat, each sensor may be calibrated individually or a number of sensors may be calibrated in a bulk;
Configuring parameters, such as but not limited to pressure and time thresholds, for different patients or for different areas on the pressure-detection mat;
Monitoring and logging a patient's pressure-relief care routine over time;
Monitoring caretakers' performances with regard to proper treatment of patients in their care;
Translating pressure sensor readings upon the sensing mat from mat coordinates to a subject's body coordinates;
Saving historical pressure data of one or more pressure-detection mat;
Allowing visual and vocal alarms through a plurality of local and mobile devices and technologies, such as but not limited to mobile phones, beepers, personal digital assistants (PDAs), display screens in nursing stations or medical carts, web interfaces, emails, Short Messaging Service (SMS), Multimedia Messaging Service (MMS), instant text messaging platforms and the like;
Allowing a patient or his caretaker to enter data with regard to patients' care status (for instance, when the patient was last moved);
Allowing for presentation, monitoring, configuration, calculation, alarms and presentation of data from multiple pressure-detection mats used by one or more subjects; and
Enabling users to query historical pressure readings and produce reports according to their needs.

External wounds caused by tissue breakdown may develop into pressure-wounds, over time. Shear forces are a common cause of such tissue breakdown. Software may further be used to analyze data received from at least one pressure detection mat and to determine whether shear forces are exerted upon body parts of a subject. Where a subject rests upon the mat, two adjacent sensors are expected to measure approximately similar pressure levels. If that is not the case, the software may deduce that the subject is sliding upon the sensing mat and shear forces are possibly exerted upon the subject's body, creating tissue breakdown.

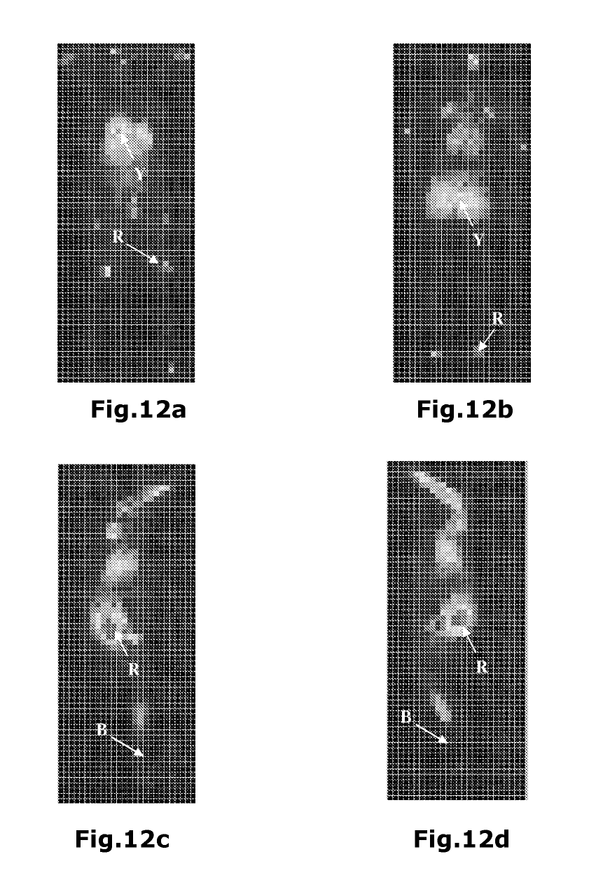

Reference is now made to FIGS. 12a-d, showing various representations of how pressure data may be displayed on a screen of an embodiment of display system 170 (FIG. 1). Respectively FIGS. 12a-d show a subject lying on his abdomen (FIG. 12a), his back (FIG. 12b), his left side (FIG. 12c) and his right side (FIG. 12d). The system shows the pressure distribution for each posture.

The display system may be a computer in communication with the data storage unit 160 (FIG. 1a), for example. Each display screen shows a matrix of pixels, each pixel representing one sensor of the pressure-detection mat. The pressure detected by each pixel is represented by a visual indication. A grayscale may be used such that higher pressures are indicated by different shades, darker grays, for example. Alternatively or additionally, colors may be used for example indicating high pressure formed between a subject's body and the surface on which the subject rests by displaying the pixel in a distinctive color, such as red (marked with R). Likewise pixels representing sensors which detect low pressure or no pressure at all may be presented in other colors such as yellow (marked with Y), blue (marked with B) or black.

Further, software may be used to analyze data received from at least one pressure detection mat and to determine a risk locus, a location where a bedsore is likely to form, for example, a location with high pressure formed between a subject's body and the surface on which the subject rests. Once the risk locus is determined, the information regarding the risk locus (e.g., the location on the mat, the magnitude of the pressure measured by the sensors at and/or near the risk locus, and the like) may be transmitted (via wire or wirelessly) to an inflatable mat including a plurality of cells operable to have its level of inflation adjusted (by, e.g., an inflation control unit). The inflatable mat may then adjust the level of inflation of cells at or near the location on the inflatable mat corresponding to the risk locus in or to redistribute the pressure at the risk locus by inflating/deflating the cells at and/or near the risk locus in one or more adjust patterns, as described herein.

Data analyzed from a pressure detection mat may be presented to at least one of a care-giver, a nurse, a man-monitored station, a friend or family member of the subject, to the subject himself or any relevant party. The display unit used to present data may be, for example and without limitation, one or more of computer screens, laptops, PDAs, cellular phone screens, printed sheets and integrated LCD screens (e.g. TFT, touch screens).

Displaying data to more than one monitor, for example both to a family member and a hired caretaker of a subject, may assist in verification that the subject is receiving proper care from his caregiver. Displaying data to the subject himself is particularly useful in paraplegic subjects who have partial mobility. For example, a subject paralyzed from the waist down and sitting in a wheelchair may not be able to sense that a pressure-wound is forming on his abdomen. However, using the pressure-wound prevention system, he can receive a notification that accumulated pressure has been detected where his abdomen typically rests. The subject may then lean his hands on the wheelchair's arm rests and lift his abdomen off the wheelchair seat for several seconds, thus relieving pressure off the sensitive area.

Data display may include alarms. Alarms may be vocal, visual, tactile, or the like. Presentation of the alarms may be ‘local’ to the subject himself or ‘remote’ when presented to one or more users typically in charge of a subject's care, such as but not limited to a family member or a nurse at a monitoring station.

The system may further be configured to include components capable of sending data regarding the system's whereabouts, using a global positioning system (GPS) or other tracking technologies as suit requirements. For example, data such as pressure-wound formation alerts may be sent along with the system's location to a manned monitoring station. This capability may be useful, for example, when data is sent to a caretaker in charge of multiple subjects who use wheelchairs for mobility within a hospital, a nursing home or another care environment. This information can assist the caretaker in finding the subject within the care facility he resides in and provide him with proper care.

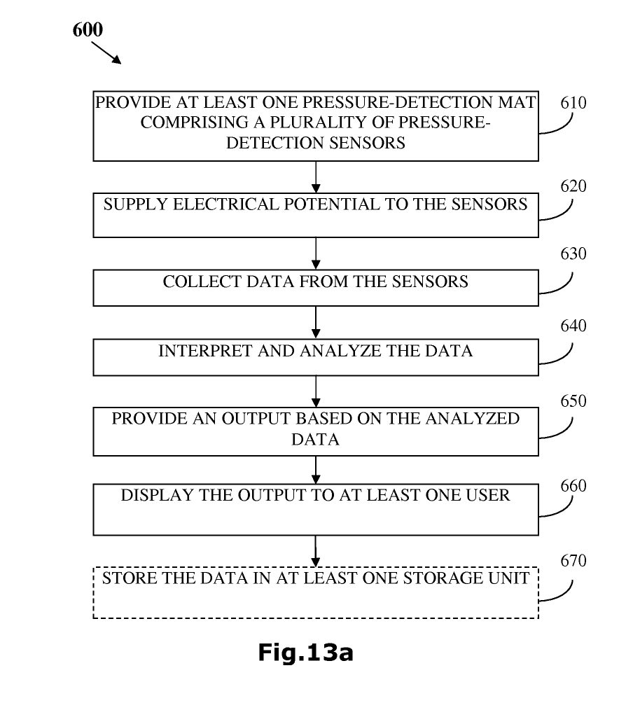

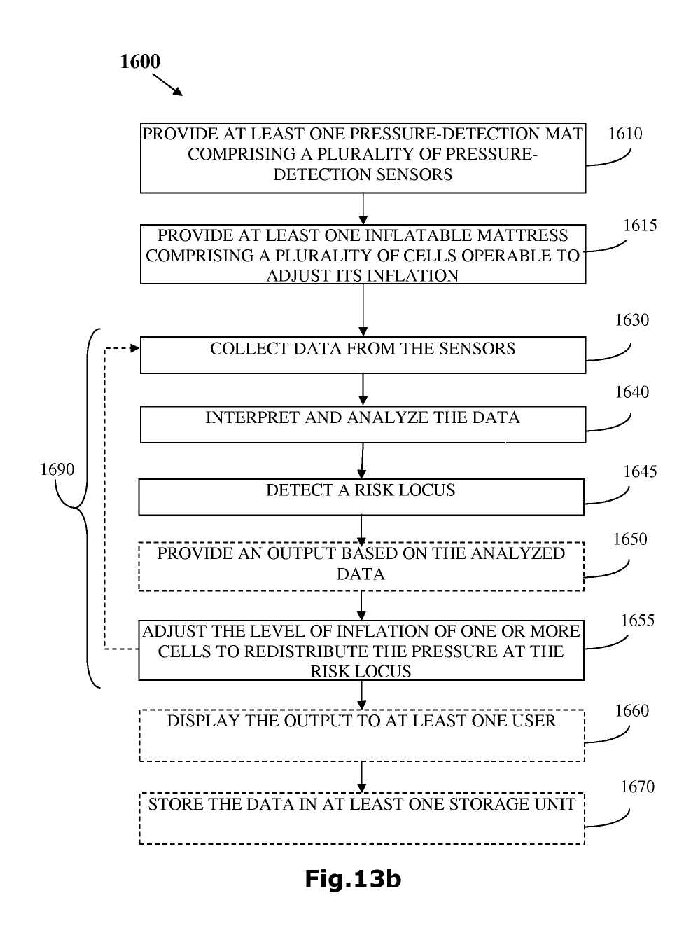

Reference is now made to FIGS. 13a-b illustrating flowcharts of methods 600 and 1600 to prevent pressure-wounds in a subject resting upon a platform. It is to be understood that unless otherwise defined, the method steps described hereinbelow can be executed either contemporaneously or sequentially in many combinations or orders of execution. Specifically, neither the ordering nor the numerals of the flowcharts of FIGS. 13a-b are to be considered as limiting. For example, two or more method steps, appearing in the following description or in the flowcharts of FIG. 13a-b in a particular order, can be executed in a different order (e.g., a reverse order) or substantially contemporaneously.

With reference to FIG. 13a, method 600 commences with providing at least one pressure-detection mat comprising a plurality of pressure-detection sensors 610. The method continues with supplying electrical potential to the sensors 620, collecting data from the sensors 630, interpreting and analyzing the data collected from the sensors 640, providing an output based upon the analyzed data 650, displaying the output to at least one user 660, and optionally storing the data in at least one data storage unit 670.

With reference to FIG. 13b, an alternative method 1600 is provided, which provides for the pressure detection mat to provide data to control an inflatable mat, The method commences with providing at least one pressure-detection mat comprising a plurality of pressure-detection sensors 1610 and providing at least one active mattress comprising a plurality of cells operable to adjust its inflation 615. The method continues with collecting data from the sensors 1630, interpreting and analyzing the data collected from the sensors 1640, detecting a risk locus 1645, and adjusting the level of inflation of one or more cells to redistribute the pressure at the risk locus 1655. Optionally, the method 1600 may contain additional steps, e.g., optionally providing an output based upon the analyzed data 1650, optionally displaying the output to at least one user 1660, and optionally storing the data in at least one data storage unit 1670. As a further option, the method 1600 may, following the step of and adjusting the level of inflation of one or more cells to redistribute the pressure at the risk locus 1655, may repeat the step of supplying electrical potential to the sensors 1610.

The cycle of risk locus detection by the pressure-detection mat and the cell inflation adjustment in the inflatable mat may be repeated to evaluate the effectiveness of the previous round of cell inflation adjustment in achieving one or more of the pressure redistribution targets and to further adjust cell inflation if needed, and/or to continue adjusting the pressure redistribution as the subject's position changes. As such, the steps of: collecting data from said plurality of sensors 1630; analyzing said data 1640; detecting a risk locus 1645; and adjusting the level of inflation of one or more cells to redistribute the pressure at the risk locus 1655 may be repeated as an adjustment cycle 1690.

The adjustment cycle 1690 may be characterized by a predetermined rate. The adjustment cycle 1690 may be repeated a predetermined number of times or a predetermined amount of time. Alternatively, the adjustment cycle 1690 may be repeated as initiated by the subject or a user such as a caregiver. Alternatively, the adjustment cycle 1690 may repeat at a predetermined rate may be started and stopped according to start/stop instructions as provided by the subject or the user.

It will be appreciated that the system as described hereinabove may be particularly useful in care facilities such as, amongst others, acute care facilities, sub-acute care facilities, long term care facilities, home care environments, hospices, hospitals, nursing homes, assisted living facilities and the like. In addition similar systems may be adapted for use in other environments such as hotels, vehicle seats, passenger seats, airplane seats, long-haul flight seats and the like.

The scope of the present invention is defined by the appended claims and includes both combinations and sub combinations of the various features described hereinabove as well as variations and modifications thereof, which would occur to persons skilled in the art upon reading the foregoing description.

Technical and scientific terms used herein should have the same meaning as commonly understood by one of ordinary skill in the art to which the disclosure pertains. Nevertheless, it is expected that during the life of a patent maturing from this application many relevant systems and methods will be developed.

As used herein the term “about” refers to at least ±10%.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. It should be understood, therefore, that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

1. A mattress adjustment system comprising:
at least one pressure detection mat comprising a plurality of sensors arranged in a matrix and at least one processor configured to monitor a plurality of pressure values detected by each of said sensors; and
at least one active mattress comprising a plurality of cells, the cells being controlled by an inflation control unit that is connected to said processor, and operable to individually adjust the level of inflation of said plurality of cells based on said plurality of pressure values.

2. The system of claim 1, wherein the pressure detection mat comprises:
at least one layer of an insulating material sandwiched between a first layer of conducting strips and a second layer of conducting strips, said conducting strips of the first layer and said strips of the second layer overlapping at a plurality of intersections, each of said sensors comprising at least one of said intersections;
a driving unit configured to supply electrical potential selectively to the conducting strips of the first layer; and
a control unit wired to the conductive strips of the said second layer and operable to control said driving unit;
the processor being further configured to monitor an electrical potential on the conductive strips of the second layer, to calculate a plurality of impedance values for each of said intersections and to determine a plurality of pressure values for each of said sensors based on said impedance values.

3. The system of claim 1, wherein:
the processor is further configured to detect a risk locus comprising an area corresponding to one or more of said sensors determined to have a pressure value beyond a predefined threshold pressure for over a predefined duration; and
the inflation control unit is operable to individually adjust the level of inflation of said plurality of cells in order to redistribute the pressure at the risk locus.

4. The system of claim 3, wherein the predefined duration is instantaneous.

5. The system of claim 3, wherein the predefined duration is dependent on the predefined threshold pressure, wherein an area corresponding to the sensor detecting a higher predefined threshold pressure requires a shorter predefined duration for the area corresponding to the sensor to be detected as a risk locus.

6. The system of claim 3, wherein said redistribution of pressure results in one or more pressure redistribution targets selected from the group consisting of:
pressure values measured by the sensors corresponding to the risk locus are lowered such that the risk locus is no longer designated as a risk locus;
the pressure measured by the sensors corresponding to the risk locus are lowered to zero; and
other loci outside the risk locus not becoming designated as a risk locus as a result of the pressure redistribution.

7. The system of claim 3, wherein said redistribution of pressure may be one or more adjustment patterns selected from the group consisting of:
reducing the pressure at the risk locus
increasing the pressure of a peripheral area neighboring the risk locus; and
reducing the pressure of a peripheral area neighboring the risk locus.

8. The system claim 7, wherein said adjustment pattern changes over time.

9. The system of claim 1, wherein each cell covers an area corresponding to the area covered by a plurality of sensors.

10. The system of claim 1, wherein the cells are identical in size and shape.

11. The system of claim 1, wherein the cells are of more than one size.

12. The system of claim 1, wherein the cells incorporated in at least one portion of the mattress designed to support a body part vulnerable to bedsore formation are smaller than the cells in other portions of the active mattress.

13. The system of claim 12, wherein said body part vulnerable to bedsore formation is one or more of a selection from the group consisting of: a sacrum, a coccyx, a heel, a hip, an elbow, a knee, an ankle or the back of the cranium.

14. A method for preventing the development of pressure-wounds comprising the steps of:
providing at least one pressure detection mat comprising a plurality of sensors configured to detect pressure;
providing at least one active mattress comprising a plurality of cells operable to adjust its inflation;
collecting data from said plurality of sensors;
analyzing said data;
detecting a risk locus; and
adjusting the level of inflation of one or more cells to redistribute the pressure at the risk locus.

15. The method of claim 14, wherein the steps of;
collecting data from said plurality of sensors;
analyzing said data;
detecting a risk locus; and
adjusting the level of inflation of one or more cells to redistribute the pressure at the risk locus
are repeated as an adjustment cycle.

16. The method of claim 15, wherein the adjustment cycle is characterized by a predetermined rate.

17. The method of claim 16, wherein the adjustment cycle is repeated for a predetermined number of times or a predetermined amount of time.

18. The method of claim 16, wherein, the adjustment cycle is started and stopped according to start/stop instructions provided by the subject or a user.

19. The method of claim 15, wherein each adjustment cycle is initiated by the subject or a user.

20. The method of claim 15, wherein said redistribution of pressure results in one or more pressure redistribution targets selected from the group consisting of:
pressure values measured by the sensors corresponding to the risk locus are lowered such that the risk locus is no longer designated as a risk locus.
the pressure measured by the sensors corresponding to the risk locus are lowered to zero.
other loci outside the risk locus do not become designated as a risk locus as a result of the pressure redistribution.

21. The method of claim 14, further comprising the step of displaying a visual output based upon said data.

22. The method of claim 14 further comprising the step of storing the data in at least one data storage unit.

23-36. (canceled)
